(19) United States
(12) Patent Application Publication
(10) Pub. No.: US 2026/0034219 A1
(43) Pub. Date: Feb. 5, 2026

(54) PROCESS FOR PREPARING SUPRAMOLECULAR CARRIER PHOTOSENSITIZER FOR PHOTODYNAMIC THERAPY AND FORMULATION THEREOF

(71) Applicant: CHANGSHA EASYINSMILE INTELLIGENCE TECHNOLOGY CO., LTD., CHANGSHA (CN)

(72) Inventors: DAN GUO, CHANGSHA (CN); TIRAN ZHANG, CHANGSHA (CN)

(73) Assignee: CHANGSHA EASYINSMILE INTELLIGENCE TECHNOLOGY CO., LTD., CHANGSHA (CN)

(21) Appl. No.: 19/260,493

(22) Filed: Jul. 5, 2025

(30) Foreign Application Priority Data
Aug. 1, 2024 (WO) PCT/CN2024/109103

(51) Int. Cl. A61K 41/00; A61K 47/56

(52) U.S. Cl. CPC A61K 41/0057; A61K 47/56

(57) ABSTRACT

A process for preparing a supramolecular carrier photosensitizer for photodynamic therapy and a formulation thereof is disclosed for prevention, treatment, anti-inflammatory and sterilization of oral dental diseases. Supramolecular carrier consists of refined lignin, glucomannan, cysteine, and sodium benzoate at a mass ratio of 0.1-1.0%: 0.5-3%: 0.1-0.5%: 0.05%, and the balance of water. Aqueous solutions of refined lignin, glucomannan, cysteine and sodium benzoate are prepared, and a pH value of combined solution is adjusted to 6.5, so that dissolved alkali lignin is precipitated to form an efficient and stable three-dimensional polymer network skeleton, and rich hydrogen bonds are facilitated to be formed between the same molecules and different molecules of lignin and glucomannan, thereby forming a supramolecular carrier formulation loaded with the photosensitizer. It ensures long-term storage stability of physicochemical traits of formulation, which is conducive to rapidly promoting application of PDT in anti-inflammatory and sterilization treatment of oral dental diseases.

### TECHNICAL FIELD

The present disclosure relates to a process for preparing a supramolecular carrier photosensitizer for photodynamic therapy and a formulation thereof, and belongs to the field of formulations for prevention, treatment, anti-inflammatory and sterilization of oral dental diseases.

### BACKGROUND

Bacterial infection is an important factor leading to the occurrence and development of inflammation of the gingival, periodontal, pulpal and periapical tissues. Due to the particularity of the intraoral environment, the composition of the pathogenic bacterial flora in oral lesion tissues are numerous and complex, and these bacteria mostly exist in the form of biofilms at lesion sites, making it very difficult to eliminate and cure them. Therefore, removing or killing pathogenic bacteria is an inevitable way to prevent and treat dental diseases. Currently, conventional treatment means for dental anti-inflammation and sterilization mainly include antibiotic treatment, surgical treatment and preoperative irrigation with a sterilization solution. Topical coating, oral, or injectable antibiotics have serious drug resistance, thus affecting the effectiveness of multiple times of treatment. Basic treatment and surgical treatment cause gingival trauma and root hypersensitivity. The commonly used preoperative irrigation with a sodium hypochlorite solution has a good sterilization effect, but the diffusion depth of the solution in the narrow part of the root canal is limited, and the cleaning and sterilization effect is restricted. Moreover, sodium hypochlorite is corrosive and cytotoxic, and easily damages soft tissues in contact with it, and even the airway.

Conventional treatment means for dental anti-inflammation and sterilization mainly include preoperative irrigation with a sterilization solution, treatment with scaling surgery and antibiotic treatment. Different from conventional treatment means, the sterilization principle of photodynamic therapy (abbreviated as: PDT) is based on the fact that in a process that a photosensitizer absorbs the energy of light of a specific wavelength and comes into an excited state, and then transitions back to a ground state, the photosensitizer releases energy to activate tissue oxygen, and uses the cytotoxicity of active oxygen to achieve anti-inflammatory and sterilization effects. Based on a large number of in vitro and in vivo evaluations, the PDT method is more effective in treating Porphyromonas gingivalis and the like. Compared with current conventional treatment means, the PDT therapy has outstanding advantages in reducing treatment damage, reducing side effects, and overcoming drug resistance. In recent years, the research on the application of PDT in the treatment of dental diseases has attracted great attention in the industry, and its clinical application has made important progress. For photosensitizer formulations as one of the key factors for PDT application, toluidine blue and a gel combined agent thereof have developed the fastest. Firstly, a toluidine blue photosensitizer has no obvious toxic side effects. Secondly, toluidine blue is an eosinophilic dye with a tendency toward nucleic acids, which has good selective adsorption on lesion areas and pathogenic bacteria, thereby preventing normal tissues from being killed by the photodynamic therapy. Thirdly, the gel formulation has certain viscosity and adhesiveness, which can constrain the photosensitizer to stay at a diseased site to improve the treatment effect, and can further ensure that the treatment process does not damage normal tissues from the perspective of dosage form. Patent CN106822894A discloses a recipe of a photosensitizing formulation for photodynamic therapy of periodontitis and use thereof, where the photosensitizing formulation is mainly consists of toluidine blue, carbomer, sodium hydroxide or triethanolamine, where carbomer is a gel component in the formulation. Patent CN108042544B discloses a toluidine blue photodynamic bactericidal and healing-promoting composition and use thereof. The composition consists of toluidine blue, sodium hyaluronate and chitosan at a ratio of 1:(0.1-2):(0.5-10), where sodium hyaluronate and chitosan are gel components in the formulation. The results of the study has showed that these combined dosage forms show excellent therapeutic effects in the prevention and treatment of periodontal and gingival diseases and sterilization of wounds after oral surgery. However, low-content and low-viscosity gels, especially physical gels, tend to become unstable due to phase separation during long-term storage caused by factors such as ambient temperature, temperature, and storage time. This is a common adverse factor that affects the qualification rate of a gel photosensitizer dosage form.

In view of the aforementioned possible problems, the present disclosure constructs an efficient and stable supramolecular network in a photosensitizer product by introducing biomass lignin rich in oxygen-containing active groups, aiming at developing a long-term stable photosensitizer formulation for gingival and periodontal localized anti-inflammatory and sterilization and a production process thereof. Thus, it ensures that problems of unqualified quality indexes such as surface crusting, flocculation and precipitation, viscosity reduction and the like do not occur during the long-term storage of the formulation.

### SUMMARY

An objective of the present disclosure is to provide a process for preparing a supramolecular carrier photosensitizer for photodynamic therapy and a formulation thereof for use in anti-inflammatory and sterilizing photodynamic therapy of oral gingiva, periodontitis, peri-implantitis and pulpitis, thereby overcoming the deficiencies in the prior art.

The technical principle of the present disclosure is that lignin is one of the components constituting a plant cell wall and has the function of connecting cells. The lignin is a biopolymer having a three-dimensional network structure formed by 3 phenylpropane units interconnected by ether bonds and carbon-carbon bonds. The molecule contains abundant active groups such as aromatic groups, phenolic hydroxyl groups, alcoholic hydroxyl groups, and carbon-based conjugated double bonds. It forms a sodium salt or potassium salt easily soluble in water under strong alkaline conditions, and under acidic conditions, a large number of hydrogen bonds are formed among molecules by substituting metal ions with H+ and then it precipitated out. The present disclosure utilizes the characteristics of a lignin alkali dissolution and acid precipitation method, and refined lignin precipitated in the pH range of 7.5 to 6.5 is preferred, of which the molecular weight distribution is more uniform. When the refined lignin is added into a formulation system, on one hand a stable three-dimensional network structure is formed under the condition of a pH value of 6.5. The stability of this three-dimensional network structure may be partially destroyed only under the condition that the pH value increases, and its stability and irreversibility are not affected by ambient temperature, temperature, storage time, etc. Also, the abundant oxygen-containing groups on the lignin form intermolecular hydrogen bonds with the abundant hydroxyl groups on the glucomannan. This ensures the long-term stability of the formulation.

The technical solution adopted by the present disclosure is as follows: a process for preparing a supramolecular carrier photosensitizer, including the following steps:
step A1. taking a specified amount of distilled water, adding a certain amount of glucomannan, and stirring at room temperature until the glucomannan is completely swollen and dispersed for later use;
step A2. taking a specified amount of distilled water, adding a certain amount of biomass-refined lignin, and adjusting a pH value to an interval of 7.5-9.0 with baking soda while stirring at room temperature, until complete dissolution to obtain a refined lignin solution for later use;
step A3. taking a specified amount of distilled water, fully dissolving a photosensitizer in the distilled water to formulate a photosensitizer aqueous solution of 200 mg/L-1,000 mg/L, then adding a certain amount of cysteine, and stirring at room temperature until complete dissolution for later use;
step A4. weighing and adding a certain amount of sodium benzoate into a specified amount of distilled water, and stirring until the sodium benzoate is completely dissolved for later use;
step A5. mixing the four solutions prepared above evenly to obtain a mixture, and adding acetic acid to adjust a pH value to 6.0-6.5; and
step A6. placing the mixture in a water bath kettle, heating to 45° C.-65° C. and keeping at this temperature for 5-48 hours, and cooling naturally to obtain a supramolecular carrier-loaded photosensitizer formulation.

The refined lignin is prepared by an alkali dissolution and acid precipitation method, wherein in the process of acid precipitation, purified lignin separated at a pH value in the interval of 7.5 to 6.5 is selected, and a specific process for preparing the refined lignin includes the following steps:
step B1. dispersing a raw material lignin in water, adding a sodium hydroxide solution while stirring to adjust the pH value to ≥11.0, and continually stirring until complete dissolution;
step B2. filtering to remove insoluble impurities;
step B3. adding a sulfuric acid solution into a filtrate while stirring to adjust the pH value to 7.5, continually stirring for a period of time, then filtering out precipitated lignin, and leaving a filtrate for later use; and
step B4. slowly adding a dilute sulfuric acid solution to the collected filtrate while stirring to adjust the pH value to 6.5, filtering, and repeatedly washing a filtered substance with distilled water and drying to obtain the refined lignin.

A raw material lignin of the refined lignin includes any one of enzymatically hydrolyzed lignin, milled wood lignin, alkali lignin, kraft lignin and the like lignin.

The photosensitizer is at least one of silica sol gel-based toluidine blue O, toluidine blue and methylene blue, and is formulated from the following components in percentage by mass: 0.001-0.1% of the toluidine blue O, 2-15% of nano silica, and the balance of normal saline;

The nano silica includes hydrophilic silica and hydrophobic silica, and the hydrophilicity and lipophilicity of the photosensitizer combined agent are regulated by adjusting the ratio of the hydrophilic silica and the hydrophobic silica.

The nano silica includes fumed silica and liquid-phase silica;
the fumed silica is of an amorphous state, and has an initial particle size between 7-40 nanometers;
the liquid phase silica has porosity between 95%-99.8% and a specific surface area>1,000 m²/g; and
the liquid-phase silica is produced by a sol-gel method, and freeze drying or supercritical drying is adopted as the mated drying process.

The method for preparing the photosensitizer includes the following steps:
step C1. fully dissolving at least one of silica sol gel-based toluidine blue O, toluidine blue and methylene blue in 85% of a specified amount of normal saline;
step C2. slowly adding a specified amount of nano silica while stirring at a stirring speed of 50-100 revolutions per minute with a high-speed disperser, and then continually stirring for 5 minutes after completion of the addition;
step C3. gradually adjusting a rotation speed to 3,000-5,000 revolutions per minute, and continually stirring in the high-speed disperser for 10 minutes to ensure uniform dispersion; and
step C4. stopping the stirring, replenishing the remaining 15% of the specified amount of normal saline, rinsing an inner wall of a container while replenishing, and then stirring at a low speed of 100 revolutions per minute for 10 minutes to obtain the photosensitizer.

A photosensitizer combined agent is further included, where the active ingredients of the photosensitizer combined agent includes a photosensitizer, a carbohydrate component, and a penetrating agent at a mass ratio of 1:(10-200):(0-5);
the photosensitizer combined agent adopts normal saline as a solvent, and a weight percentage of the active ingredients is 0.5% to 5%;
the photosensitizer combined agent has viscosity of 5 to 5,000 mPa·s.

A method for preparing the photosensitizer combined agent includes the following steps:
step D1. fully dissolving a photosensitizer in normal saline;
step D2. adding a carbohydrate component, dissolving or swelling, and stirring to mix evenly;
step D3. adding a penetrating agent, and mixing evenly under a condition of stirring; and
step D4. making up with normal saline to a total mass percentage of 100%, and stirring thoroughly to obtain a final combined agent.
when applied for photodynamic anti-inflammatory and sterilization treatment of root canal, the combined agent is a low-viscosity formulation with viscosity between 3 to 100 mPa·s, including 3 mPa·s and 100 mPa·s; and more preferably between 5 to 10 mPa·s;
when applied for photodynamic anti-inflammatory and sterilization treatment of a periodontal pocket, the combined agent is a medium-viscosity formulation with viscosity between 100 to 1,800 mPa·s, including 1,800 mPa s; and more preferably between 500 to 1,000 mPa s; and
when applied for photodynamic anti-inflammatory and sterilization treatment of a gingival surface, the combination is applied to photodynamic anti-inflammatory and sterilization treatment on the gingival surface, the combined agent is a high-viscosity formulation with viscosity between 1,800 to 5,000 mPa s, including 5,000 mPa·s; and more preferably between 2,500 to 3,000 mPa·s.

The supramolecular carrier formulation consists of: refined lignin, glucomannan, cysteine, sodium benzoate at a mass ratio of 0.1-1.0%: 0.5-3%: 0.1-0.5%: 0.05-0.2%, and the balance of water.

The beneficial effects of the present disclosure are: The supramolecular structure carrier of this formulation is made of lignin and glucomannan in combination and in cooperation, and takes advantage of the fact that the efficient three-dimensional supramolecular network structure constructed by the refined lignin is not easy to be deteriorated as affected by storage conditions such as temperature, humidity, time, and light as well as environmental changes, thereby ensuring the long-term stability of the physicochemical traits of the carrier in the photosensitive formulation. It overcomes the disadvantage that the long-term storage stability of ordinary gel formulations, especially low-content and low-viscosity physical gel formulations, is easily affected by factors such as ambient temperature, temperature, and storage time. Compared with ordinary gel-loaded photosensitizer formulations, the formulation of the present disclosure not only has the advantages of the ordinary gel formulations, but also can ensure the long-term storage stability of the physicochemical traits of the formulation. The product qualification rate is a key factor in determining the application of the formulation. Therefore, the present disclosure is conducive to quickly promoting the application of PDT in oral anti-inflammatory and sterilization treatment.

### BRIEF DESCRIPTION OF THE DRAWINGS

The accompanying drawings illustrate one or more embodiments of the present disclosure, and features and benefits thereof, and together with the written description, serve to explain the principles of the present invention. Wherever possible, the same reference numbers are used throughout the drawings to refer to the same or like elements of an embodiment, and wherein:

FIG. 1 shows a flowchart of a process for preparing a supramolecular carrier photosensitizer according to certain embodiments of the present disclosure;

FIG. 2 shows a flowchart of a process for refining lignin according to certain embodiments of the present disclosure;

FIG. 3 shows a flowchart of a method for preparing the photosensitizer according to certain embodiments of the present disclosure; and

FIG. 4 shows a flowchart of a method for preparing the photosensitizer combined agent according to certain embodiments of the present disclosure.

### DETAILED DESCRIPTION OF THE EMBODIMENTS

In order to make the objectives, technical solutions and advantages of the present disclosure clearer, the present disclosure is further described in detail hereafter in conjunction with detailed description and with reference to the accompanying drawings. It should be understood that these descriptions are exemplary only and are not intended to limit the scope of the present disclosure. Furthermore, in the following description, descriptions of well-known structures and technologies are omitted to avoid unnecessarily obscuring the concepts of the present disclosure.

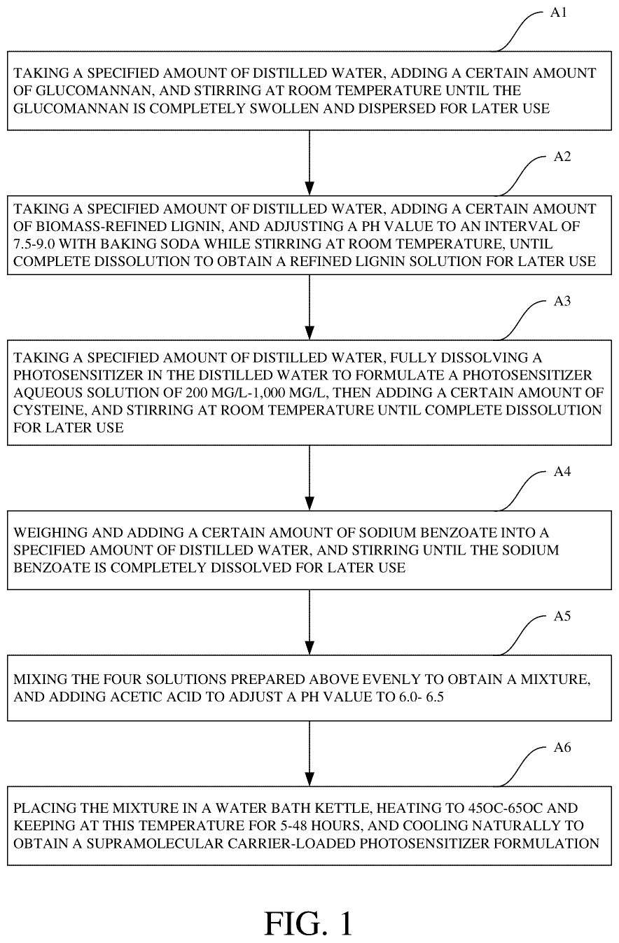

Referring now to FIG. 1, a process for preparing a supramolecular carrier photosensitizer is shown according to certain embodiments of the present disclosure. The technical solution adopted by the present disclosure is as follows: a process for preparing a supramolecular carrier photosensitizer, including the following steps:
STEP A1. taking a specified amount of distilled water, adding a certain amount of glucomannan, and stirring at room temperature until the glucomannan is completely swollen and dispersed for later use;
STEP A2. taking a specified amount of distilled water, adding a certain amount of biomass-refined lignin, and adjusting a pH value to an interval of 7.5-9.0 with baking soda while stirring at room temperature, until complete dissolution to obtain a refined lignin solution for later use;
STEP A3. taking a specified amount of distilled water, fully dissolving a photosensitizer in the distilled water to formulate a photosensitizer aqueous solution of 200 mg/L-1,000 mg/L, then adding a certain amount of cysteine, and stirring at room temperature until complete dissolution for later use;
STEP A4. weighing and adding a certain amount of sodium benzoate into a specified amount of distilled water, and stirring until the sodium benzoate is completely dissolved for later use;
STEP A5. mixing the four solutions prepared above evenly to obtain a mixture, and adding acetic acid to adjust a pH value to 6.0-6.5; and
STEP A6. placing the mixture in a water bath kettle, heating to 45° C.-65° C. and keeping at this temperature for 5-48 hours, and cooling naturally to obtain a supramolecular carrier-loaded photosensitizer formulation.

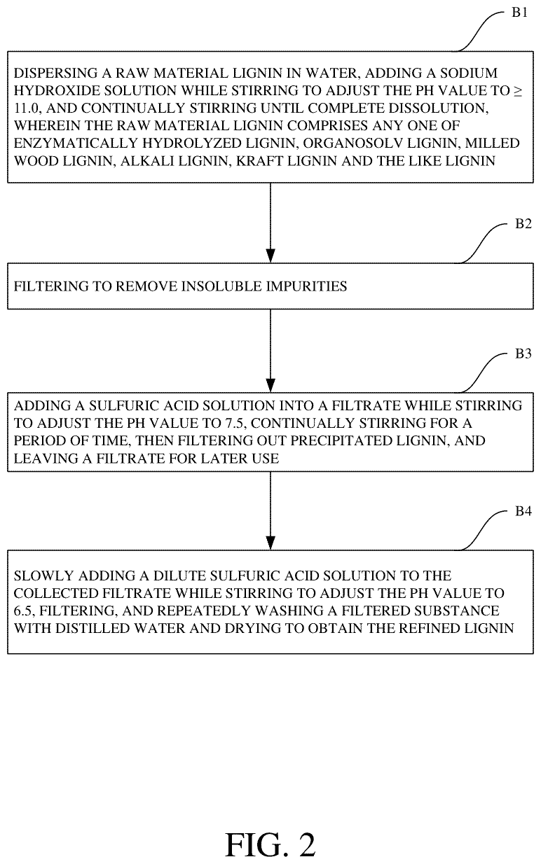

Referring now to FIG. 2, a process for a process for refining lignin is shown according to certain embodiments of the present disclosure. The refined lignin is prepared by an alkali dissolution and acid precipitation method, wherein in the process of acid precipitation, purified lignin separated at a pH value in the interval of 7.5 to 6.5 is selected, and a specific process for preparing the refined lignin includes the following steps:
STEP B1. dispersing a raw material lignin in water, adding a sodium hydroxide solution while stirring to adjust the pH value to ≥11.0, and continually stirring until complete dissolution;
STEP B2. filtering to remove insoluble impurities;
STEP B3. adding a sulfuric acid solution into a filtrate while stirring to adjust the pH value to 7.5, continually stirring for a period of time, then filtering out precipitated lignin, and leaving a filtrate for later use; and
STEP B4. slowly adding a dilute sulfuric acid solution to the collected filtrate while stirring to adjust the pH value to 6.5, filtering, and repeatedly washing a filtered substance with distilled water and drying to obtain the refined lignin.

A raw material lignin of the refined lignin includes any one of enzymatically hydrolyzed lignin, milled wood lignin, alkali lignin, kraft lignin and the like lignin.

The photosensitizer is at least one of silica sol gel-based toluidine blue O, toluidine blue and methylene blue, and is formulated from the following components in percentage by mass: 0.001-0.1% of the toluidine blue O, 2-15% of nano silica, and the balance of normal saline;

The nano silica includes hydrophilic silica and hydrophobic silica, and the hydrophilicity and lipophilicity of the photosensitizer combined agent are regulated by adjusting the ratio of the hydrophilic silica and the hydrophobic silica.

The nano silica includes fumed silica and liquid-phase silica;
the fumed silica is of an amorphous state, and has an initial particle size between 7-40 nanometers;
the liquid phase silica has porosity between 95%-99.8% and a specific surface area>1,000 m²/g; and
the liquid-phase silica is produced by a sol-gel method, and freeze drying or supercritical drying is adopted as the mated drying process.

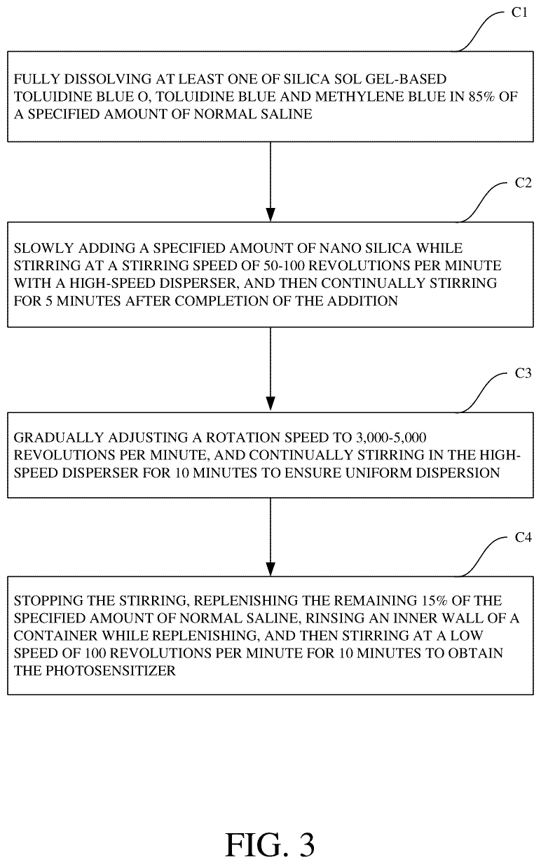

Referring now to FIG. 3, a method for preparing the photosensitizer is shown according to certain embodiments of the present disclosure. The method for preparing the photosensitizer includes the following steps:
STEP C1. fully dissolving at least one of silica sol gel-based toluidine blue O, toluidine blue and methylene blue in 85% of a specified amount of normal saline;
STEP C2. slowly adding a specified amount of nano silica while stirring at a stirring speed of 50-100 revolutions per minute with a high-speed disperser, and then continually stirring for 5 minutes after completion of the addition;
STEP C3. gradually adjusting a rotation speed to 3,000-5,000 revolutions per minute, and continually stirring in the high-speed disperser for 10 minutes to ensure uniform dispersion; and
STEP C4. stopping the stirring, replenishing the remaining 15% of the specified amount of normal saline, rinsing an inner wall of a container while replenishing, and then stirring at a low speed of 100 revolutions per minute for 10 minutes to obtain the photosensitizer.

A photosensitizer combined agent is further included, where the active ingredients of the photosensitizer combined agent includes a photosensitizer, a carbohydrate component, and a penetrating agent at a mass ratio of 1:(10-200):(0-5);
the photosensitizer combined agent adopts normal saline as a solvent, and a weight percentage of the active ingredients is 0.5% to 5%;
the photosensitizer combined agent has viscosity of 5 to 5,000 mPa·s.

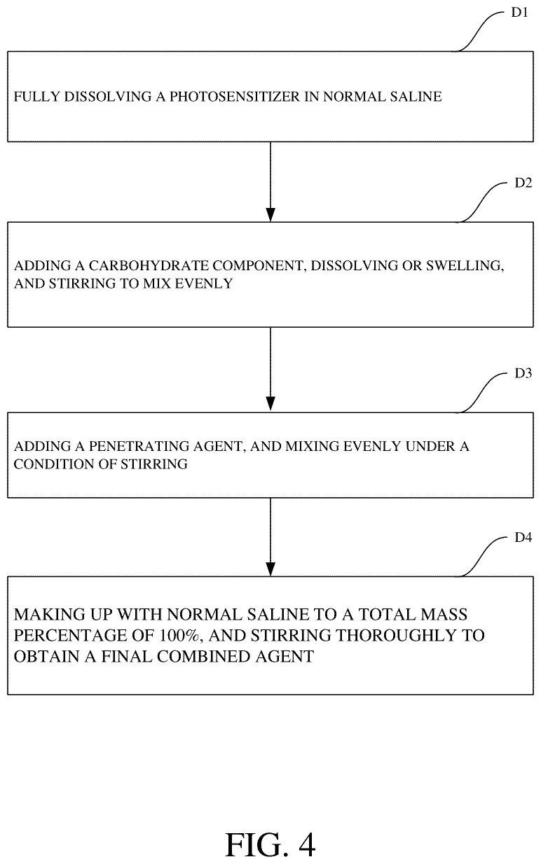

Referring now to FIG. 4, a method for preparing the photosensitizer combined agent is shown according to certain embodiments of the present disclosure. The method for preparing the photosensitizer combined agent includes the following steps:
STEP D1. fully dissolving a photosensitizer in normal saline;
STEP D2. adding a carbohydrate component, dissolving or swelling, and stirring to mix evenly;
STEP D3. adding a penetrating agent, and mixing evenly under a condition of stirring; and
STEP D4. making up with normal saline to a total mass percentage of 100%, and stirring thoroughly to obtain a final combined agent.
when applied for photodynamic anti-inflammatory and sterilization treatment of root canal, the combined agent is a low-viscosity formulation with viscosity between 3 to 100 mPa·s, including 3 mPa·s and 100 mPa·s; and more preferably between 5 to 10 mPa·s;
when applied for photodynamic anti-inflammatory and sterilization treatment of a periodontal pocket, the combined agent is a medium-viscosity formulation with viscosity between 100 to 1,800 mPa·s, including 1,800 mPa·s; and more preferably between 500 to 1,000 mPa·s; and
when applied for photodynamic anti-inflammatory and sterilization treatment of a gingival surface, the combination is applied to photodynamic anti-inflammatory and sterilization treatment on the gingival surface, the combined agent is a high-viscosity formulation with viscosity between 1,800 to 5,000 mPa·s, including 5,000 mPa s; and more preferably between 2,500 to 3,000 mPa·s.

The supramolecular carrier formulation consists of: refined lignin, glucomannan, cysteine, sodium benzoate at a mass ratio of 0.1-1.0%: 0.5-3%: 0.1-0.5%: 0.05-0.2%, and the balance of water.

### Example 1

Step 1. 100 mL of distilled water was measured, then added with 2.0 g of glucomannan, and stirred at room temperature for 24 hours until the glucomannan was completely swollen and dispersed. The resulting solution was named 1-A.
Step 2. 90 mL of distilled water was taken, added with 1.2 g of refined lignin, adjusted to a pH value in an interval of 7.5-8.0 with a baking soda solution while stirring at room temperature, continually stirred until the lignin is completely dispersed and dissolved, and then added with distilled water to a volume of 100 mL. The resulting solution is named 1-B.
Step 3. 100 mL of distilled water was taken, and 20 mg of toluidine blue O was weighed and fully dissolved in the distilled water, then added with 0.4 g of cysteine, and stirred at room temperature until it was completely dissolved. The resulting solution was named 1-C.
Step 4. 0.2 g of sodium benzoate was weighed and added into distilled water to formulate a 100 mL solution, and the solution was magnetically stirred until the sodium benzoate was completely dissolved. The resulting solution was named 1-D.
Step 5. the solution 1-B was added into the solution 1-A while stirring, the mixture was stirred evenly and then added with the solutions 1-C and 1-D, the mixture was stirred evenly and then added with acetic acid to adjust the pH value to 6.5. The resulting intermediate sample was named 1-E.
Step 6. the intermediate sample 1-E was heated in a water bath kettle to 45° C., kept at this temperature for 20 hours, and then cooled naturally to obtain the supramolecular carrier photosensitizer formulation.

The composition ratio of the components in the formulation was toluidine blue O, refined lignin, glucomannan, cysteine, and sodium benzoate at a mass ratio of 0.005%: 0.3%: 0.5%: 0.1%: 0.05%, and the balance of water.

### Example 2

The specific preparation process was as shown in Example 1. The composition ratio of the components in the formulation was the toluidine blue O, the refined lignin, the glucomannan, the cysteine, the sodium benzoate at a mass ratio of 0.005%: 0.3%: 1.0%: 0.1%: 0.05%, and the balance of water.

### Example 3

The specific preparation process was as shown in Example 1. The composition ratio of the components in the formulation was the toluidine blue O, the refined lignin, the glucomannan, the cysteine, the sodium benzoate at a mass ratio of 0.005%: 0.6%: 0.5%: 0.1%: 0.05%, and the balance of water.

### Example 4

The specific preparation process was as shown in Example 1. The composition ratio of the components in the formulation was the toluidine blue, the refined lignin, the glucomannan, the cysteine, the sodium benzoate at a mass ratio of 0.005%: 0.6%: 0.5%: 0.1%: 0.05%, and the balance of water.

### Example 5

The specific preparation process was as shown in Example 1. The composition ratio of the components in the formulation was the methylene blue, the refined lignin, the glucomannan, the cysteine, the sodium benzoate at a mass ratio of 0.005%: 0.6%: 0.5%: 0.1%: 0.05%, and the balance of water.

The comparison in performances and effects of the comparative example to Example 5 was shown in Table 1:

TABLE 1
Comparison in performances and effects

| | | Lignin | | Stability | | |
|---|---|---|---|---|---|---|
| | | content | Glucomannan | 1 | 3 | 6 |
| Examples | Photosensitizer | (%) | content (%) | month | months | months |
| Comparative | Toluidine blue O | 0 | 0.5 | 100% | 50% | 20% |
| Example | | | | | | |
| Example 1 | Toluidine blue O | 0.3 | 0.5 | 100% | 100% | 80% |
| Example 2 | Toluidine blue O | 0.3 | 1.0 | 100% | 100% | 100% |
| Example 3 | Toluidine blue O | 0.6 | 0.5 | 100% | 100% | 100% |
| Example 4 | Toluidine blue | 0.6 | 0.5 | 100% | 100% | 100% |
| Example 5 | Methylene blue | 0.6 | 0.5 | 100% | 100% | 100% |

### Table 1 Comparison in Performances and Effects

Stability test: The formulation obtained in each comparative example and example was packed into 10 small samples, and the small samples were packed in 50 mL transparent glassy cylindrical sample bottles of the same specifications, placed in a natural environment and stored with protection from light, and the stability of the samples was observed after 1 month, 3 months, and 6 months. If there was surface conjunctiva, flocculation and precipitation, physical phase separation, viscosity decrease, etc., it was judged as an abnormal product. The percentage of normal small samples among the 10 small samples was used for representing the stability of the formulation.

Apparently, the described embodiments are merely a part rather than all of the embodiments of the present disclosure. All other embodiments obtained by those of ordinary skills in the art based on the embodiments of the present invention without creative efforts shall fall within the claimed scope of the present invention.

What is claimed is:

1. A process for preparing a supramolecular carrier photosensitizer for photodynamic therapy, comprising:
STEP A1. taking a specified amount of distilled water, adding a certain amount of glucomannan, and stirring at room temperature until the glucomannan is completely swollen and dispersed for later use;
STEP A2. taking a specified amount of distilled water, adding a certain amount of biomass-refined lignin, and adjusting a pH value to an interval of 7.5-9.0 with baking soda while stirring at room temperature, until complete dissolution to obtain a refined lignin solution for later use;
STEP A3. taking a specified amount of distilled water, fully dissolving a photosensitizer in the distilled water to formulate a photosensitizer aqueous solution of 200 mg/L-1,000 mg/L, then adding a certain amount of cysteine, and stirring at room temperature until complete dissolution for later use;
STEP A4. weighing and adding a certain amount of sodium benzoate into a specified amount of distilled water, and stirring until the sodium benzoate is completely dissolved for later use;
STEP A5. mixing the four solutions prepared above evenly to obtain a mixture, and adding acetic acid to adjust a pH value to 6.0-6.5; and
STEP A6. placing the mixture in a water bath kettle, heating to 45° c.-65° c. and keeping at this temperature for 5-48 hours, and cooling naturally to obtain a supramolecular carrier-loaded photosensitizer formulation.

2. The process for preparing a supramolecular carrier photosensitizer for photodynamic therapy according to claim 1, wherein:
the refined lignin is prepared by an alkali dissolution and acid precipitation method, wherein in the process of acid precipitation, purified lignin separated at a pH value in the interval of 7.5 to 6.5 is selected, and a specific preparation process of the refined lignin is as follows:
STEP B1. dispersing a raw material lignin in water, adding a sodium hydroxide solution while stirring to adjust the pH value to ≥11.0, and continually stirring until complete dissolution, wherein the raw material lignin comprises any one of enzymatically hydrolyzed lignin, organosolv lignin, milled wood lignin, alkali lignin, kraft lignin and the like lignin;
STEP B2. filtering to remove insoluble impurities;
STEP B3. adding a sulfuric acid solution into a filtrate while stirring to adjust the pH value to 7.5, continually stirring for a period of time, then filtering out precipitated lignin, and leaving a filtrate for later use; and
STEP B4. slowly adding a dilute sulfuric acid solution to the collected filtrate while stirring to adjust the pH value to 6.5, filtering, and repeatedly washing a filtered substance with distilled water and drying to obtain the refined lignin.

3. The process for preparing a supramolecular carrier photosensitizer for photodynamic therapy according to claim 1, wherein:
a raw material lignin of the refined lignin comprises any one of enzymatically hydrolyzed lignin, milled wood lignin, alkali lignin, kraft lignin and the like lignin.

4. The process for preparing a supramolecular carrier photosensitizer for photodynamic therapy according to claim 1, wherein the photosensitizer is at least one of silica sol gel-based toluidine blue O, toluidine blue and methylene blue, and is formulated from the following components in percentage by mass: 0.001-0.1% of the toluidine blue O, 2-15% of nano silica, and the balance of normal saline;
the nano silica comprises hydrophilic silica and hydrophobic silica, and the hydrophilicity and lipophilicity of the photosensitizer combined agent are regulated by adjusting the ratio of the hydrophilic silica and the hydrophobic silica.

5. The process for preparing a supramolecular carrier photosensitizer for photodynamic therapy according to claim 4, wherein the nano silica comprises fumed silica and liquid-phase silica;
the fumed silica is of an amorphous state, and has an initial particle size between 7-40 nanometers;
the liquid phase silica has porosity between 95%-99.8% and a specific surface area>1,000 m²/g; and
the liquid-phase silica is produced by a sol-gel method, and freeze drying or supercritical drying is adopted as the mated drying process.

6. The process for preparing a supramolecular carrier photosensitizer for photodynamic therapy according to claim 4, comprising the following steps:
STEP C1. fully dissolving at least one of silica sol gel-based toluidine blue O, toluidine blue and methylene blue in 85% of a specified amount of normal saline;
STEP C2. slowly adding a specified amount of nano silica while stirring at a stirring speed of 50-100 revolutions per minute with a high-speed disperser, and then continually stirring for 5 minutes after completion of the addition;
STEP C3. gradually adjusting a rotation speed to 3,000-5,000 revolutions per minute, and continually stirring in the high-speed disperser for 10 minutes to ensure uniform dispersion; and
STEP C4. stopping the stirring, replenishing the remaining 15% of the specified amount of normal saline, rinsing an inner wall of a container while replenishing, and then stirring at a low speed of 100 revolutions per minute for 10 minutes to obtain the photosensitizer.

7. The process for preparing a supramolecular carrier photosensitizer for photodynamic therapy according to claim 6, further comprising a photosensitizer combined agent, wherein the active ingredients of the photosensitizer combined agent consist of a photosensitizer, a carbohydrate component, and a penetrating agent at a mass ratio of 1:(10-200):(0-5);
the photosensitizer combined agent adopts normal saline as a solvent, and a weight percentage of the active ingredients is 0.5% to 5%;
the photosensitizer combined agent has viscosity of 5 to 5,000 mPa·s.

8. The process for preparing a supramolecular carrier photosensitizer for photodynamic therapy according to claim 7, wherein a method for preparing the photosensitizer combined agent comprises the following steps:
STEP D1. fully dissolving a photosensitizer in normal saline;
STEP D2. adding a carbohydrate component, dissolving or swelling, and stirring to mix evenly;
STEP D3. adding a penetrating agent, and mixing evenly under a condition of stirring; and
STEP D4. making up with normal saline to a total mass percentage of 100%, and stirring thoroughly to obtain a final combined agent.

9. The process for preparing a supramolecular carrier photosensitizer for photodynamic therapy according to claim 8, wherein:
when applied for photodynamic anti-inflammatory and sterilization treatment of root canal, the combined agent is a low-viscosity formulation with viscosity between 3 to 100 mPa·s, comprising 3 mPa·s and 100 mPa·s; and more preferably between 5 to 10 mPa·s;
when applied for photodynamic anti-inflammatory and sterilization treatment of a periodontal pocket, the combined agent is a medium-viscosity formulation with viscosity between 100 to 1,800 mPa·s, comprising 1,800 mPa·s; and more preferably between 500 to 1,000 mPa·s; and
when applied for photodynamic anti-inflammatory and sterilization treatment of a gingival surface, the combination is applied to photodynamic anti-inflammatory and sterilization treatment on the gingival surface, the combined agent is a high-viscosity formulation with viscosity between 1,800 to 5,000 mPa·s, comprising 5,000 mPa·s; and more preferably between 2,500 to 3,000 mPa·s.

10. A supramolecular carrier formulation in a process for preparing a supramolecular carrier photosensitizer for photodynamic therapy, consisting of: refined lignin, glucomannan, cysteine, sodium benzoate at a mass ratio of 0.1-1.0%: 0.5-3%: 0.1-0.5%: 0.05-0.2%, and the balance of water, wherein the process for preparing a supramolecular carrier photosensitizer for photodynamic therapy comprises:
STEP A1. taking a specified amount of distilled water, adding a certain amount of glucomannan, and stirring at room temperature until the glucomannan is completely swollen and dispersed for later use;
STEP A2. taking a specified amount of distilled water, adding a certain amount of biomass-refined lignin, and adjusting a pH value to an interval of 7.5-9.0 with baking soda while stirring at room temperature, until complete dissolution to obtain a refined lignin solution for later use;
STEP A3. taking a specified amount of distilled water, fully dissolving a photosensitizer in the distilled water to formulate a photosensitizer aqueous solution of 200 mg/L-1,000 mg/L, then adding a certain amount of cysteine, and stirring at room temperature until complete dissolution for later use;
STEP A4. weighing and adding a certain amount of sodium benzoate into a specified amount of distilled water, and stirring until the sodium benzoate is completely dissolved for later use;
STEP A5. mixing the four solutions prepared above evenly to obtain a mixture, and adding acetic acid to adjust a pH value to 6.0-6.5; and
STEP A6. placing the mixture in a water bath kettle, heating to 45° c.-65° c. and keeping at this temperature for 5-48 hours, and cooling naturally to obtain a supramolecular carrier-loaded photosensitizer formulation.

11. The supramolecular carrier formulation according to claim 10, wherein the refined lignin is prepared by an alkali dissolution and acid precipitation method, wherein in the process of acid precipitation, purified lignin separated at a pH value in the interval of 7.5 to 6.5 is selected, and a specific preparation process of the refined lignin is as follows:
STEP B1. dispersing a raw material lignin in water, adding a sodium hydroxide solution while stirring to adjust the pH value to ≥11.0, and continually stirring until complete dissolution, wherein the raw material lignin comprises any one of enzymatically hydrolyzed lignin, organosolv lignin, milled wood lignin, alkali lignin, kraft lignin and the like lignin;
STEP B2. filtering to remove insoluble impurities;
STEP B3. adding a sulfuric acid solution into a filtrate while stirring to adjust the pH value to 7.5, continually stirring for a period of time, then filtering out precipitated lignin, and leaving a filtrate for later use; and
STEP B4. slowly adding a dilute sulfuric acid solution to the collected filtrate while stirring to adjust the pH value to 6.5, filtering, and repeatedly washing a filtered substance with distilled water and drying to obtain the refined lignin.

12. The supramolecular carrier formulation according to claim 10, wherein a raw material lignin of the refined lignin comprises any one of enzymatically hydrolyzed lignin, milled wood lignin, alkali lignin, kraft lignin and the like lignin.

13. The supramolecular carrier formulation according to claim 10, wherein the photosensitizer is at least one of silica sol gel-based toluidine blue O, toluidine blue and methylene blue, and is formulated from the following components in percentage by mass: 0.001-0.1% of the toluidine blue O, 2-15% of nano silica, and the balance of normal saline;
the nano silica comprises hydrophilic silica and hydrophobic silica, and the hydrophilicity and lipophilicity of the photosensitizer combined agent are regulated by adjusting the ratio of the hydrophilic silica and the hydrophobic silica.

14. The supramolecular carrier formulation according to claim 13, wherein the nano silica comprises fumed silica and liquid-phase silica;
the fumed silica is of an amorphous state, and has an initial particle size between 7-40 nanometers;
the liquid phase silica has porosity between 95%-99.8% and a specific surface area>1,000 m²/g; and
the liquid-phase silica is produced by a sol-gel method, and freeze drying or supercritical drying is adopted as the mated drying process.

15. The supramolecular carrier formulation according to claim 13, wherein the process for preparing a supramolecular carrier photosensitizer for photodynamic therapy comprises the following steps:
STEP C1. fully dissolving at least one of silica sol gel-based toluidine blue O, toluidine blue and methylene blue in 85% of a specified amount of normal saline;
STEP C2. slowly adding a specified amount of nano silica while stirring at a stirring speed of 50-100 revolutions per minute with a high-speed disperser, and then continually stirring for 5 minutes after completion of the addition;
STEP C3. gradually adjusting a rotation speed to 3,000-5,000 revolutions per minute, and continually stirring in the high-speed disperser for 10 minutes to ensure uniform dispersion; and
STEP C4. stopping the stirring, replenishing the remaining 15% of the specified amount of normal saline, rinsing an inner wall of a container while replenishing, and then stirring at a low speed of 100 revolutions per minute for 10 minutes to obtain the photosensitizer.

16. The supramolecular carrier formulation according to claim 15, wherein the process for preparing a supramolecular carrier photosensitizer for photodynamic therapy further comprises a photosensitizer combined agent, wherein the active ingredients of the photosensitizer combined agent consist of a photosensitizer, a carbohydrate component, and a penetrating agent at a mass ratio of 1:(10-200):(0-5);
the photosensitizer combined agent adopts normal saline as a solvent, and a weight percentage of the active ingredients is 0.5% to 5%;
the photosensitizer combined agent has viscosity of 5 to 5,000 mPa·s.
